(19) United States
(12) Patent Application Publication
(10) Pub. No.: US 2013/0017274 A1
(43) Pub. Date: Jan. 17, 2013

(54) LOW DOSE LITHIUM IN THE TREATMENT OR PROPHYLAXIS OF PARKINSON'S DISEASE

(71) Applicants: JULIE K. ANDERSEN, Novato, CA (US); HWAN KIM, San Francisco, CA (US)

(72) Inventors: JULIE K. ANDERSEN, Novato, CA (US); HWAN KIM, San Francisco, CA (US)

(73) Assignee: BUCK INSTITUTE FOR RESEARCH ON AGING, Novato, CA (US)

(21) Appl. No.: 13/422,960

(22) Filed: Mar. 16, 2012

Related U.S. Application Data
(60) Provisional application No. 61/453,477, filed on Mar. 16, 2011.

(51) Int. Cl. A61K 33/14; A61P 25/16

(52) U.S. Cl. 424/677

(57) ABSTRACT

In various embodiments methods of delaying the onset of and/or mitigating the severity of one or more symptoms of Parkinson's disease in a mammal are provided. In certain embodiments the methods involve administering to a mammal diagnosed as having or as at risk for Parkinson's disease a chronic low dose of lithium (e.g., a subtherapeutic dose). In certain embodiments the low dose lithium is administered in conjunction with another agent (e.g., L-DOPA).

### CROSS-REFERENCE TO RELATED APPLICATIONS

This application claims benefit of and priority to U.S. Ser. No. 61/453,477, filed on Mar. 16, 2011, which is incorporated herein by reference in its entirety for all purposes.

### STATEMENT OF GOVERNMENTAL SUPPORT

[Not Applicable]

### BACKGROUND OF THE INVENTION

Aside from Alzheimer's disease, Parkinson's disease is the most well-known disease in the neurodegenerative disease group. Parkinson's disease (PD) is a chronic and progressive degenerative disease of the brain that impairs motor control, speech, and other functions. In particular, it is characterized by (1) a slowing down of all movements (bradykinesia), quiet and monotonous speech (akinesia or hypokinesia), absence of the physiological associated movements, a stooped posture, a small-step, partially shuffling gait, handwriting which becomes smaller as the writing continues, uncontrollable disturbances in movement, with a tendency to fall forward to the side or backward, (2) rigidity of the musculature (rigor), and (3) coarse resting tremor (trembling). Parkinson's disease is a disease that occurs relatively frequently and develops in approx. 1% of individuals aged over 60, in particular in men. The disease is caused by loss of dopamine in the striatum, resulting in the degeneration of neurons in the substantia nigra. The primary reason for loss of dopamine is not known (Dunnett and Bjorklund (1999) Nature 399: A32-A39; Olanow and Tatton (1999) Annu Rev. Neurosci. 22: 123-144).

The motor symptoms of PD are caused by loss of nerve cells that secrete dopamine in a tiny midbrain area called the substantia nigra. For reasons that are not fully understood, these nerve cells are especially vulnerable to damage of various sorts, including drugs, disease, and head trauma. The term Parkinsonism is used for any process that destroys large numbers of these cells and thereby causes the same characteristic symptoms. Parkinson's disease, or more fully, idiopathic Parkinson's disease, is typically diagnosed when no specific physical cause for the loss of dopamine cells can be identified.

The term Parkinsonism is used for symptoms of tremor, stiffness, and slowing of movement caused by loss of dopamine cells in the substantia nigra. “Parkinson's disease” is the synonym of “primary Parkinsonism”, i.e. isolated Parkinsonism due to a neurodegenerative process without any secondary systemic cause. In some cases, it would be inaccurate to say that the cause is “unknown” because a small proportion is caused by identifiable genetic mutations. T is possible for a patient to be initially diagnosed with Parkinson's disease but then to develop additional features requiring revision of the diagnosis.

There are other disorders called Parkinson-plus diseases. These include: multiple system atrophy (MSA), progressive supranuclear palsy (PSP), corticobasal degeneration (CBD), and dementia with Lewy bodies (DLB). Lewy bodies are abnormal aggregates of protein that develop inside nerve cells. Most idiopathic Parkinson's disease patients also have Lewy bodies in their brain tissue, but the distribution is denser and more widespread in DLB. Even so, the relationship between Parkinson's disease, Parkinson's disease with dementia, and dementia with Lewy bodies (DLB) might be most accurately conceptualized as a spectrum, with a discrete area of overlap between each of the three disorders.

The Parkinson-plus diseases may progress more quickly than typical idiopathic Parkinson disease. If cognitive dysfunction occurs before or very early in the course of the movement disorder then DLB may be suspected. Early postural instability with minimal tremor especially in the context of ophthalmoparesis should suggest PSP. Early autonomic dysfunction including erectile dysfunction and syncope may suggest MSA. The presence of extreme asymmetry with patchy cortical cognitive defects such as dysphasia and apraxias especially with “alien limb” phenomena should suggest CBD.

### SUMMARY

In various embodiments methods of slowing the onset and/or inhibiting the onset and/or severity of one or more symptoms of Parkinson's Disease and/or a “Parkinsons-plus” disease are provided. In certain embodiments the methods involve the administration of low dose lithium alone, or in combination with one or more pharmacological agents (e.g., L-DOPA).

In various embodiments lithium and/or L-DOPA is provided in a form that is not a naturally occurring form, and/or a nutritional or dietary supplement. In various embodiments lithium is provided in a form that is not lithium orotate.

In various embodiments methods of slowing the onset and/or inhibiting the onset and/or severity of one or more symptoms of Parkinson's Disease and/or a “Parkinsons-plus” disease are provided. In certain embodiments the methods involve the administration of low dose lithium alone, or in combination with one or more pharmacological agents (e.g., L-DOPA).

In various embodiments lithium and/or L-DOPA is provided in a form that is not a naturally occurring form, and/or a nutritional or dietary supplement. In various embodiments lithium is provided in a form that is not lithium orotate.

In various embodiments a method of delaying the onset of and/or mitigating the severity of one or more symptoms of Parkinson's disease in a mammal is provided. The method typically involves administering to a mammal diagnosed as having or as at risk for Parkinson's disease a chronic low dose of lithium. In certain embodiments the low dose is a subtherapeutic dose. In certain embodiments the low dose is a low therapeutic dosage. In certain embodiments the low dose is a dosage that results in an average serum concentration below about 0.5 mM or 0.4 mM, or below about 0.3, mM, or below about 0.2 mM, or below about 0.1 mM. In certain embodiments the dosage ranges from about 0.05 mM, or 0.1 mM to about 0.2 mM, or 0.3 mM, or 0.4 mM, or 0.5 mM. In certain embodiments the mammal is a human. In various embodiments human is not under treatment for a neuropsychiatric disorder. In various embodiments the human is not under treatment for one or more disorders selected from the group consisting of mania, schizophrenia, bipolar disorder, and psychosis. In certain embodiments the mammal is a human diagnosed as having Parkinson's disease. In certain embodiments the mammal is a human diagnosed as at risk for Parkinson's disease. In certain embodiments the human is a human 40 years of age or older, or 45 years of age or older, or 50 years of age or older, or 55 years of age or older, or 60 years of age or older, or 65 years of age or older, or 70 years of age or older, or 75 years of age or older. In certain embodiments the administration is before the onset of cardinal motor symptoms of Parkinson's disease. In certain embodiments the administration is to a subject diagnosed as having olfactory dysfunction. In certain embodiments the human is diagnosed with one or more conditions selected from the group consisting of tremor, bradykinesia, and muscle rigidity.

Methods are also provided for delaying the onset of and/or mitigating the severity of one or more symptoms of Parkinson's disease in a mammal, where the methods typically involve administering to a mammal determined to have or to be at risk for Parkinson's disease a chronic low dose of lithium in conjunction with one or more pharmaceutically active agents selected from the group consisting of L-3,4-dihydroxyphenylalanine (L-DOPA) and/or a derivative thereof, a MAOB inhibitor, and a dopamine agonist. In certain embodiments the second agent comprises L-DOPA or a derivative thereof. In certain embodiments the second agent comprises a L-DOPA/carbidopa combination. In certain embodiments the lithium is administered prior to second agent or after the second agent, or simultaneously with the second agent. In certain embodiments the lithium and the second agent(s) are administered in a combined formulation. In certain embodiments the method further comprises administering the lithium, and L-DOPA in conjunction with a DOPA decarboxylase inhibitor and/or a COMT inhibitor and/or pyridoxal phosphate. In certain embodiments the lithium is administered as a lithium salt. In certain embodiments the L-DOPA is provided as an L-DOPA ethyl ester. In certain embodiments the L-DOPA is provided as a combined L-DOPA/carbidopa (e.g., SINEMET®) formulation. In certain embodiments the L-DOPA is administered in a sub-therapeutic dose for Parkinson's disease. In certain embodiments the method comprises administering a peripheral DOPA decarboxylase inhibitor (DDCI). In certain embodiments the DDCI is selected from the group consisting of benserazide, (25)-3-(3,4-dihydroxyphenyl)-2-hydrazino-2-methylpropanoic acid (carbidopa), and methyldopa. In certain embodiments the L-DOPA and DDCI (e.g., carbidopa) are provided as a combined formulation. In certain embodiments the method comprises administering a catechol-O-methyl transferase (COMT) inhibitor (e.g., entacapone, tolcapone, nitecapone, and the like). In certain embodiments the second agent comprises a MAOB inhibitor (e.g., selegiline, desmethylselegiline, pargyline, rasagiline, AGN-1135, MDL 72195, J 508, lazabemide, milacemide, mofegiline, D-deprenyl, ladostigil, and the like). In certain embodiments the MAOB inhibitor is administered in a sub-therapeutic dose for Parkinson's disease. In certain embodiments the second agent comprises a dopamine agonist (e.g., bromocriptine, cabergoline, pergolide, pramipexole, ropinirole, piribedil, apomorphine, rigotine, quinagolide, fenoldopam, lisuride, and the like). In certain embodiments the dopamine agonist is administered in a sub-therapeutic dose for Parkinson's disease. In certain embodiments the lithium is administered at a chronic low dose. In certain embodiments the low dose is a subtherapeutic dose. In certain embodiments the low dose is a low therapeutic dosage. In certain embodiments the low dose is a dosage that results in an average serum concentration below about 0.5 mM, or below about 0.4 mM, or below about 0.3, mM, or below about 0.2 mM, or below about 0.1 mM. In certain embodiments the dosage ranges from about 0.05 mM, or 0.1 mM to about 0.2 mM, or 0.3 mM, or 0.4 mM, or 0.5 mM. In various embodiments the mammal is a human. In certain embodiments the human is not under treatment for a neuropsychiatric disorder. In certain embodiments the human is not under treatment for one or more disorders selected from the group consisting of mania, schizophrenia, bipolar disorder, and psychosis. In certain embodiments the mammal is a human diagnosed as having Parkinson's disease. In certain embodiments the mammal is a human diagnosed as at risk for Parkinson's disease. In certain embodiments the human is a human 40 years of age or older, or 45 years of age or older, or 50 years of age or older, or 55 years of age or older, or 60 years of age or older, or 65 years of age or older, or 70 years of age or older, or 75 years of age or older. In certain embodiments the administration is before the onset of cardinal motor symptoms of Parkinson's disease. In certain embodiments the administration is to a subject diagnosed as having olfactory dysfunction. In certain embodiments the human is diagnosed with one or more conditions selected from the group consisting of tremor, bradykinesia, and muscle rigidity.

In certain embodiments a formulation for delaying the onset of and/or mitigating the severity of one or more symptoms of Parkinson's disease is provided. The formulation typically comprises lithium; L-DOPA and/or a derivative thereof; and one or more pharmaceutically acceptable excipients. In certain embodiments the formulation further comprises a DOPA decarboxylase inhibitor and/or a COMT inhibitor, and/or pyridoxal phosphate. In certain embodiments the lithium a lithium salt. In certain embodiments the L-DOPA is provided as an L-DOPA ethyl ester. In certain embodiments the formulation further comprises a peripheral DOPA decarboxylase inhibitor (DDCI). In certain embodiments the DDCI is selected from the group consisting of benserazide, carbidopa, and methyldopa. In certain embodiments the formulation comprises carbidopa. In certain embodiments the formulation comprises a catechol-O-methyl transferase (COMT) inhibitor. In certain embodiments the COMT inhibitor is selected from the group consisting of entacapone, tolcapone, and nitecapone. In certain embodiments the COMT inhibitor is entacapone.

### DEFINITIONS

As used herein, “administering” refers to local and/or systemic administration, e.g., including enteral and parenteral administration. Routes of administration for the active agent(s) described herein include, but are not limited to oral administration, administration as a suppository, topical contact, intravenous administration, intraperitoneal administration, intramuscular administration, intralesional administration, nasal administration, subcutaneous administration, the implantation of a slow-release and/or regulated release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, ionophoretic and intracranial. Other modes of delivery include, but are not limited to, the use of liposomal formulations, intravenous infusion, transdermal patches, etc.

In certain embodiments administering can include causing to be administered for example by prescribing for a subject, annotating a patient's medical record, instructing an agent or other person to administer, and the like.

The terms “systemic administration” and “systemically administered” refer to a method of administering one or more compound(s) or composition(s) to a mammal so that the compound(s) or composition(s) are delivered to sites in the body, including the targeted site of pharmaceutical action, via, for example, the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The phrase “effective amount” means a dosage or dosage regimen sufficient to produce a desired result.

The term “coadministering” or “concurrent administration”, when used, for example with respect to the active agent(s) described herein (e.g., lithium and L-DOPA, lithium and L-DOPA/carbidopa, etc.) refers to administration of the active agents such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, coadministering typically results in both agents being simultaneously present in the body (e.g. in the plasma) at a significant fraction (e.g. 20% or greater, preferably 30% or 40% or greater, more preferably 50% or 60% or greater, most preferably 70% or 80% or 90% or greater) of their maximum serum concentration for any given dose.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than the active agents described herein (e.g., serotonin receptor antagonists such as loxapine and/or loxapine analogues, and/or cyproheptadine and/or cyproheptadine analogues).

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments the subject may not be under the care or prescription of a physician or other health worker.

A “subtherapeutic dosage” refers to a dosage that is below that typically used for the subject agent in typical therapeutic or prophylactic use. Thus, for example, lithium dosages typically range from a serum concentration of 0.4 mM to about 1.6 mM. A subtherapeutic dosage in this context typically results in serum concentrations below 0.4 mM. In certain embodiments the low-therapeutic or subtherapeutic dose is defined with respect to the condition being treated. Thus, for example, a subtherapeutic dose of an agent (e.g., lithium, MAOB inhibitors, dopamine agonists, etc.) for Parkinson's disease is a dose lower than that typically prescribed for Parkinson's disease relying, e.g., on an additive or synergistic effect with another agent to facilitate the desired effect.

When a Markush Group or a list of particular compounds is described in the specification and/or claims it is intended that in various additional or alternative embodiments any subset of that Markush group or list is contemplated. Thus, for example, a Markush group or list consisting of elements A, B, and C also comprises a disclosure of a Markush Group or list consisting of A, and B, a Markush Group or list consisting of B, and C, and a Markush Group or list consisting of A and C as well as elements A, B, and C individually. Similarly, when elements in one list or Markush group are described as being in combination with elements in another list or Markush group the combination of any one element of one group with any one element of another group is contemplated. Thus where a list or Markush Group consisting of A, B and C is disclosed in combination with another list or Markush Group consisting of D, E, and F, the description is to be recognized as contemplating the combinations A/D, A/E, A/F, B/D, B/E, B/F, C/D, C/E, C/F and/or any combination of A, B, or C with any subgroup or member of D, E, and F, and/or any combination of D, E, or F, with any subgroup or member of A, B, and C.

### BRIEF DESCRIPTION OF THE DRAWINGS

FIG. 1, panels A-G shows that lithium prevents oxidized/nitrated alpha-synuclein accumulation in multiple brain regions. Examples are shown of OB (panels A, C and E) and striatum (panels B, D and F). Mean density±SEM after background correction (a.u.: arbitrary unit) (panel G). One-way ANOVA, the Dunnett's post hoc test (n=6 each); *, p<0.05, **, p<0.01, n.s., not significant compared to no lithium group+PQ/MB (black bars). Cont: no lithium without PQ/MB injection. Size bar: 250 μm. Abb.: GL, glomerular layer; GCL, granule cell later; St, striatum; SNpc, Substantia Nigra pars compacta; HP, hippocampus; Cx, cortex.

FIG. 2, panels A-G, shows that lithium prevents oxidized/nitrated alpha-synuclein accumulation (oxi/nit α-Syn, 16 kD) in multiple brain regions of neuronal human A53T mutant expressing mice chronically exposed to PQ/MB. Examples of Western blot analysis of high salt (panel A) and RIPA (panel B) soluble extracts are displayed and densitometric analyses in oxi/nit α-syn Western blots shown in BS (panel C), OB (panel D), Cx (panel E), Cb (panel F), and St (panel G) of high salt samples, normalized for β-actin loading. *, p<0.05, **, p<0.01 versus vehicle with PQ/MB (black bars) as assessed by one-way ANOVA, the Dunnett's post hoc test (n=3-6 per group). Abb.: BS, Brain stem; OB, olfactory bulb; Cx, cortex; Cb, cerebellum; St, striatum; Li, lithium treatment; PQ/MB, paraquat/maneb treated group.

FIG. 3, panels A-G, shows that lithium prevents oxidized/nitrated alpha-synuclein accumulation in mitral (arrows in panels A and D) and Purkinje cells (arrows in panels B and E). Panels A, B, and C: vehicle-treated group; Panels D, E, and F: lithium-treated group. Panels C and F are enlargements of panels B and E, respectively. Panel G: Oxidized/nitrated alpha-synuclein positive cells were stereologically counted and reported as mean±SEM (n=6-7 each). **, p<0.01 versus controls as assessed by paired t-test. Bar: 75 μm.

FIG. 4, panels A-I, shows that lithium protects against neuronal cell death in several brain regions. Mitral cells (panels A and D) and Purkinje cells (panels B and E) were specifically labeled with anti-5HT-2A receptor antibody and SNpc DA neurons (panels C and F) with anti-TH antibody. Total cell numbers were stereologically counted following specific cell labeling (panel G, mitral cells; H, Purkinje cells; panel I, SNpc DA cells) and reported as mean±SEM (n=6-7 each). *, p<0.05, **, p<0.01 versus vehicle control as assessed by paired t-test. Bar: 100 μm.

FIG. 5 shows hindlimb clasping studies.

FIG. 6 shows results of a hindlimb clasping test as a function of treatment. Three days of tests were combined.

FIG. 7 shows an analysis of the hindlmib clasping tests for each treatment. The average instances of hindlimb clasping are shown for each treatment. Clasping counted for 10 seconds for 3 consecutive days was combined for data analysis (Mean±SEM, unpaired t-test: *: p<0.05, **: p<0.01, n=7, 9, 6, 6, and 9 in order above).

FIG. 8A illustrates striatum samples taken for measurement of dopamine levels within the striatum via tyrosine hydroxylase (TH) staining. FIG. 8B shows TH+ staining in the striatum for four treatments (no MPTP-saline, MPTP-saline, MPTP-lithium, and MPTP-lithium+SINEMET®).

FIG. 9 illustrates the effect of treatment on TH+Density in ST. Results are evaluated using a one way ANOVA, Dunnett's test: *: p<0.05, **: p<0.01, compared with MPTP+Sal (#), n=6-8 each group.

FIG. 10 illustrates the effect of treatment on TH+ cell count in SNpc. One way ANOVA, Dunnett's test: *: p<0.05, **: p<0.01, compared with MPTP+Sal (#), n=6-8 each group.

FIG. 11 shows the results of an N27 AT-cell, 20 S proteasomal assay. One way ANOVA, Dunnett's test: *: p<0.05, **: p<0.01, compared with No Li+MPP+(↓), n=6-8 each group.

### DETAILED DESCRIPTION

In various embodiments methods of slowing the onset and/or inhibiting the onset and/or severity of one or more symptoms of Parkinson's Disease and/or a “Parkinsons-plus” disease are provided. In certain embodiments the methods involve the administration of low dose lithium alone, or in combination with one or more pharmacological agents (e.g., L-DOPA).

In particular it is believed that chronic low dosage administration of lithium alone, or in combination with certain other agents affords a neuroprotective effect and can be used to inhibit the onset and/or rate of progression and/or severity of one or more symptoms of Parkinson's disease and/or of a Parkinson's-plus condition.

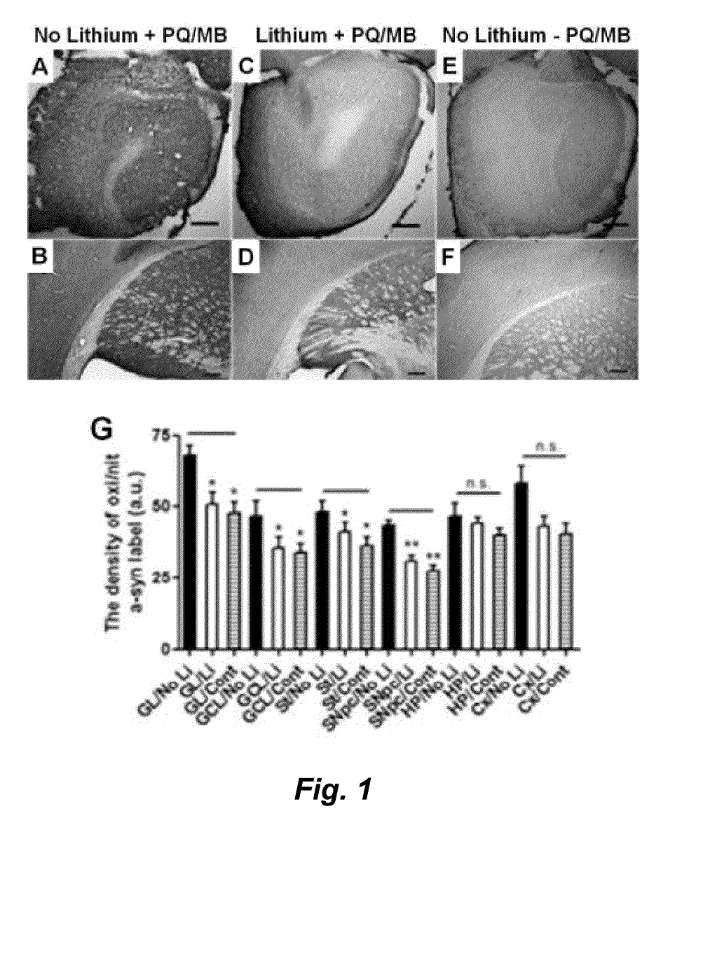

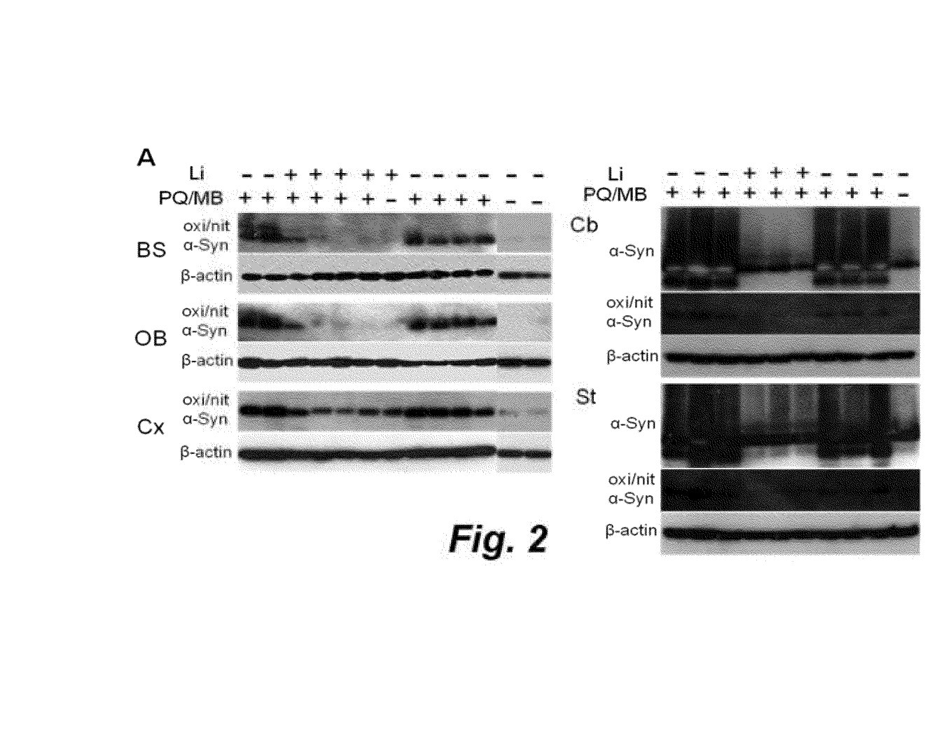

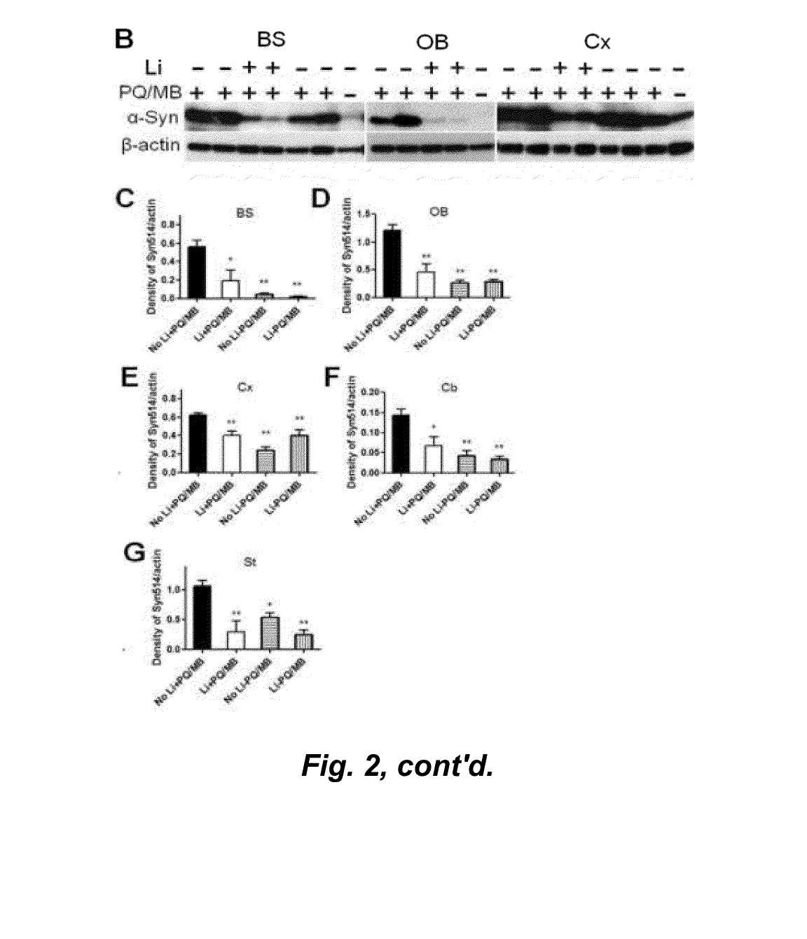

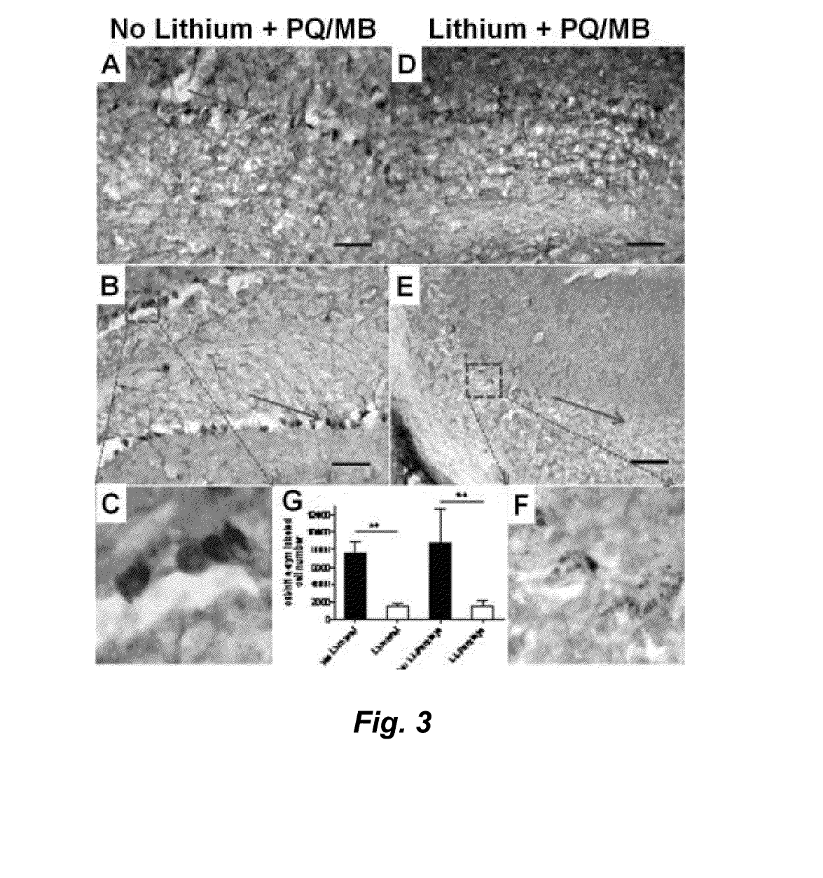

Our recent data indicate that that chronic lithium administration can prevent both oxidative stress-induced alpha-synuclein accumulation and neuronal cell death in PD models at dosages in the low therapeutic range and it is believed in the sub-therapeutic range. Lithium administration was initially found to protect against in vitro cell death in a hydrogen peroxide treated, stable human alpha-synuclein expressing dopaminergic cell line recently created by our laboratory. In vivo lithium administration (0.255% lithium chloride) delivered in feed to 9 month old pan-neuronal human alpha-synuclein expressing transgenic mice over a 3 month period was sufficient to prevent accumulation of oxidized/nitrated alpha-synuclein in multiple brain regions previously shown to occur as a consequence of paraquat/maneb administration in these transgenics. This included in the glomerular layer, mitral cells, and the granule cell layer of the olfactory bulb (OB), the striatum (ST), the substantia nigra pars compacta (SNpc), in Purkinje cells of the cerebellum (Cb), and the cortex (FIGS. 1-3).

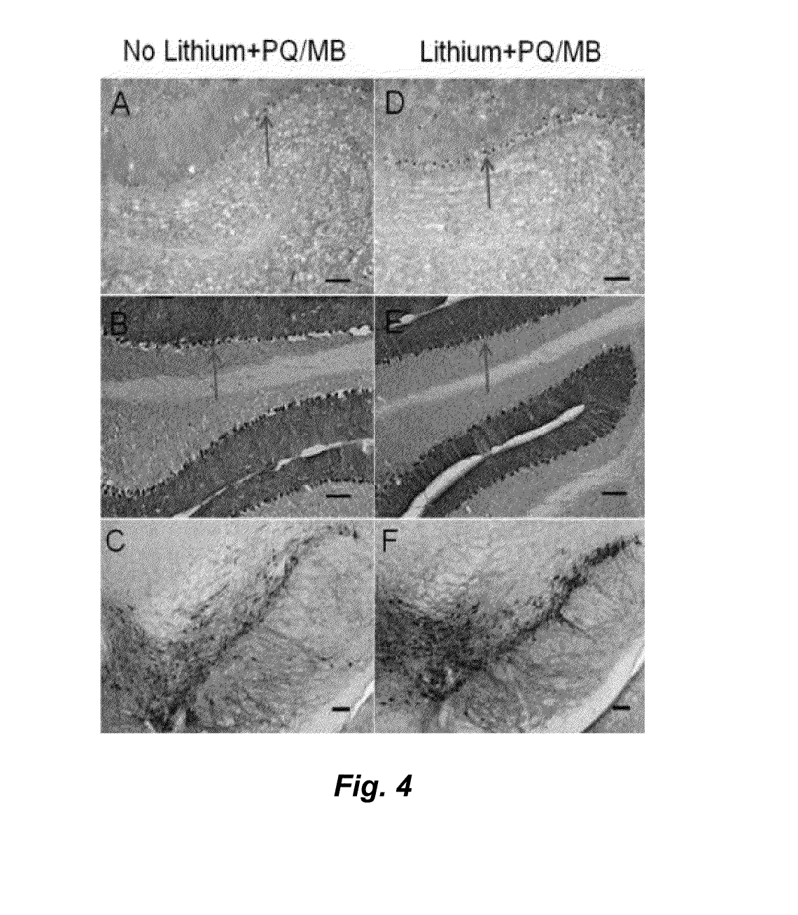

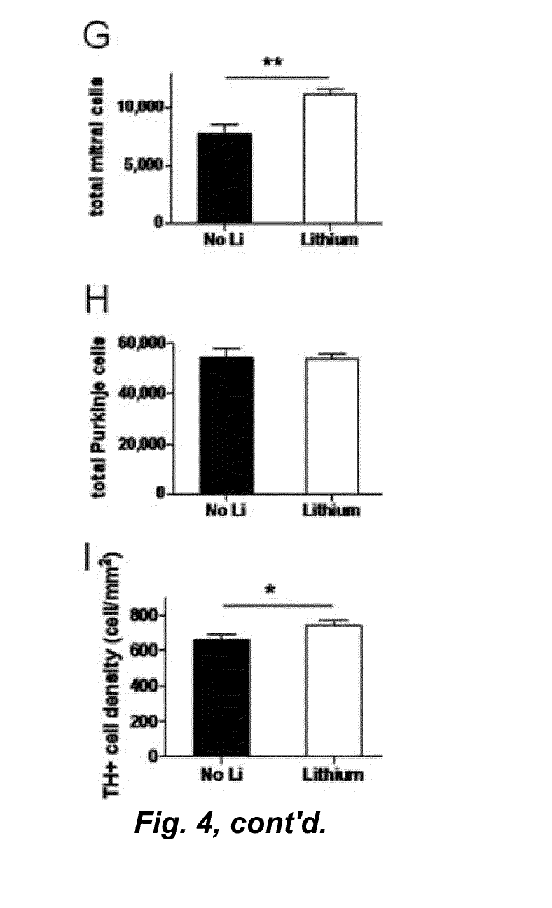

Lithium was found to not only prevent alpha-synuclein accumulation in various brain regions, but also to protect against neuronal cell loss in this model (FIG. 4). Importantly, this dosage has previously been demonstrated to result in sera lithium levels in the low therapeutic range (0.65-0.8 mM) in mice independent of strain with no effect on motor function.

In addition, low dose lithium when used in combination with SINEMET® was shown provide a synergistic improvement in locomotor behavior compared to SINEMET® alone. Moreover this improvement does not appear to be associated with dyskinesias observed at higher lithium dosages previously used in humans. Accordingly it is believed that lithium used in combination with L-DOPA and/or a levodopa/carbidopa combination can show substantially improved long term benefit to a subject under such treatment.

As indicated above, in view of these, and other data, it is believed that chronic administration of lithium in low therapeutic doses or sub-therapeutic dosages can be used be used to inhibit the onset and/or rate of progression and/or severity of one or more symptoms of Parkinson's disease and/or of a Parkinson's-plus condition. It is also contemplated that the chronic low-therapeutic or sub-therapeutic dose lithium can be used in conjunction with certain other agents (e.g., L-DOPA and/or derivatives thereof) to achieve improved efficacy in the treatment or prophylaxis of Parkinson's disease or a Parkinson's-plus condition.

In addition, combined formulations are contemplated, especially (but not limited to) combination formulations of lithium and levodopa/carbidopa (e.g., SINEMET®) combinations.

### Pharmaceutical Formulation.

In various embodiments the methods described herein contemplate administering low dosage (e.g., low therapeutic dosage, or sub-therapeutic dosage) lithium alone for the treatment or prophylaxis of Parkinson's disease or in combination with one or more additional agents. Such additional agents include, but are not limited to L-DOPA, L-carbidopa, L-DOPA/carbidopa combinations, DOPMAOB inhibitors, dopamine agonists, and the like. Particularly where the second agent comprises L-DOPA or a derivative thereof administration of additional agents can include administration of one or more DOPA decarboxylase inhibitors (DDCIs) and/or one or more catechol-O-methyl transferase (COMT) inhibitors

Lithium.

Lithium is available in a wide number of pharmaceutical formulations. Typically, lithium is provided as a salt e.g., lithium carbonate (Li₂Co₃), lithium citrate (Li₃C₆H₅O₇), lithium sulfate (Li₂SO₄), lithium chloride (LiCl), and the like.

In various embodiments the lithium can be provided as an orally administered formulation, e.g., fluid, caplet, capsule, gelcap, tablet, and the like, as an injectable, as a depot formulation, as a timed-release or slow-release formulation, as an inhalable composition, and the like.

In certain embodiments, the lithium will be provided at a dosage that produces a low therapeutic serum concentration. In certain embodiments the low-therapeutic dosage will be a dosage that provides an average serum concentration ranging from about 0.4 mM to about 1.2 mM, preferably from about 0.4 mM to about 1.0 mM, more preferably from about 0.4 mM to about 0.8 mM, and most preferably from about 0.4 mM to about 0.6 mM.

In certain embodiments the lithium is provided as a subtherapeutic dosage. An illustrative sub-therapeutic dosage produces a serum lithium concentration ranging from about 0.05 mM up to about 0.39 mM. In certain embodiments the sub-therapeutic dosage produces a serum lithium concentration range from a low of about 0.1 mM, or about 0.15 mM, or about 0.2 mM, to a high of about 0.3 mM, or about 0.34 mM, or about 0.46 mM, or about 0.39 mM.

The particular dosage administered to a subject can vary with the formulation. Typically the serum lithium is monitored in the subject according to methods well known to those of skill in the art until the optimal administered dosage is identified.

Lithium is also available as a nutritional supplement in the form of lithium orotate. In certain embodiments the use of lithium orotate is expressly excluded.

L-3,4-dihydroxyphenylalanine (L-DOPA).

In certain embodiments the chronic low dosage lithium can be administered in conjunction with one or additional agents. One such agent comprises L-3,4-dihydroxyphenylalanine (L-DOPA) and/or a derivative thereof. Illustrative L-DOPA derivatives include, but are not limited to L-DOPA amides (see, e.g., Zhou et al. (2010) Eur. J. Med. Chem., 45(9):4035-4542, which is incorporated herein by reference for the compounds described therein), L-DOPA esters (see, e.g., PCT Publication WO/1986/004579, which is incorporated herein by reference for the compounds described therein), glycosyl derivatives of L-DOPA (see, e.g., Bonina, et al. (2003) J. Drug Targeting, 11(1): 25-36, which is incorporated herein by reference for the compounds described therein), hydrogen sulfide-releasing L-DOPA derivatives (see, e.g., Lee et al. (2010) J. Biol. Chem., 285(23): 17318-17328, which is incorporated herein by reference for the compounds described therein), and the like.

In certain embodiments the L-DOPA can be administered in standard therapeutic dosages for PD. In certain embodiments the L-DOPA can be administered in a low therapeutic dose.

In certain embodiments it is believed that the low dose lithium can act additively or even synergistically with the L-DOPA. Thus, in certain embodiments it is contemplated that the L-DOPA can be administered in sub-therapeutic doses. In certain embodiments L-DOPA is administered in a subtherapeutic dosage of less than 0.5 mg/day, or less than about 0.4 mg/day, or less than about 0.3 mg/day, or less than about 0.2 mg/day.

Dopa Decarboxylase Inhibitors (DDCIs)

In certain embodiments, particularly where the L-DOPA/lithium are administered in conjunction with the lithium administration of one or more additional agents in conjunction with the L-DOPA are contemplated. Such agents include, for example, one or more additional agents include, a DOPA decarboxylase inhibitor (DDCI) or aromatic L-amino acid decarboxylase inhibitor (DDCI), and/or a catechol-O-methyl transferase (COMT) inhibitor.

In various embodiments peripheral DDCIs incapable of crossing the blood-brain-barrier (BBB) can be used in conjunction with the L-DOPA to block the peripheral conversion of L-DOPA into dopamine and thereby reduce adverse side effects. Illustrative DDCIs include, but are not limited to benserazide (e.g., MADOPAR®, PROLOPA®, MODOPAR®, MADOPARK®, NEODOPASOL®, EC-DOPARYL®, etc.), carbidopa (e.g., LODOSYN®, SINEMET®, PARCOPA®, ATAMET®, STALEVO®, etc.), methyldopa (e.g., ALDOMET®, ALDORIL®, DOPAMET®, DOPEGYT®, etc.), and the like.

A typical illustrative dosage of combinations of L-DOPA and DDCIs (e.g., SINEMET®, etc.) is SINEMET® 25:100 t.i.d. with 100 mg of levodopa TID (300 mg per day). A typical maintenance dose is between about 300 mg/day up to 800 mg 1-dopa per day. The bottom of the therapeutic range is 280 mg/day of SINEMET® (L-DOPA). Accordingly, in certain embodiments, a subtherapeutic dosage is below 280 mg/day, preferably below about 270 mg/day, or below about below about 260 mg/day, or below about 250 mg/day, or below about 240 mg/day, or below about 230 mg/day, or below about 220 mg/day, or below about 210 mg/day, or below about 200 mg/day.

Catechol-O-Methyl Transferase (COMT) Inhibitors

In various embodiments the L-DOPA/lithium are administered in conjunction with a COMT inhibitor. A COMT inhibitor is n agent that inhibits the action of catechol-O-methyl transferase. This enzyme is involved in degrading neurotransmitters. Illustrative pharmaceutical examples include, but are not limited to entacapone, tolcapone, and nitecapone.

It is noted that all of the foregoing active agents are commercially available as pharmaceutical formulations and, in certain embodiments, the use of such formulations is contemplated.

MAOB Inhibitors.

In certain embodiments administration of one or more MAOB inhibitors in conjunction with lithium is contemplated for the treatment or prophylaxis of PD. In certain embodiments selective MAOB inhibitors are contemplated, while in other embodiments, non-selective MAOB inhibitors are contemplated. Illustrative MAOB inhibitor(s) include, but are not limited to selegiline (e.g., DEPRENYL®, ELDEPRYL®, EMSAM®), JUMEX®, JUMEXAL® CARBEX®, ELDEPRYL®, MOVERGAN®; APTAPRYL®, ANIPRYL®; ELDEPRINE®; PLURIMEN®), desmethylselegiline, pargyline (EUDATIN®, SUPIRDYL®, EUTONYL®, see, e.g., U.S. Pat. No. 3,155,584, which is incorporated herein by reference), rasagiline [R(+)N-propargyl-laminoindan] (e.g., AZILECT®), 3-N-phenylacetylamino-2,5-piperidinedione, caroxyazone, AGN-1135 (see, e.g., WO 92/21333), MDL 72195 (see, e.g., WO 92/21333), J 508 (see, e.g., WO 92/21333), lazabemide (e.g., PAKIO®, TEMPIUM®, also see, e.g., WO 2000/045846), milacemide (see, e.g., WO 00/45846), IFO (see, e.g., WO 00/45846], mofegiline (see, e.g., WO 00/45846), and 5-(4-(4,4,4-trifluorobut-oxy)phenyl)-3-(2-methoxyethyl)-1,3,4-oxadiazol-2(3H)-one (see, e.g., PCT/FR2000/000193 (WO 2000/045846)), D-deprenyl, ladostigil, milacemide, mofegiline, and the like. In certain embodiments, prodrugs or metabolites of the MAOB inhibitors are contemplated. Typically the metabolite has substantially the same or better selective MAO-B inhibitor activity as its unmetabolized form.

In certain embodiments, a prodrug of a MAOB inhibitor comprises a derivatized MAOB inhibitor that is metabolized in vivo into the active inhibitory agent. Prodrugs typically have substantially the same or better therapeutic value than the underivatized MAOB inhibitor(s).

In certain embodiments the MAOB inhibitor can be administered in standard therapeutic dosages for PD. In certain embodiments the MAOB inhibitor can be administered in a low therapeutic doses.

In certain embodiments it is believed that the low dose lithium can act additively or even synergistically with the MAOB inhibitor(s). Thus, in certain embodiments it is contemplated that the MAOB inhibitor(s) can be administered in sub-therapeutic doses.

Illustrative doses for MAOB inhibitors are for rasagiline monotherapy 1 mg daily in combination with L-Dopa 0.5-1.0 mg daily. Accordingly, in certain embodiments, subtherapeutic MAOB inhibitor (rasagiline) dose is less than 0.5 mg/day, or less than about 0.4 mg/day, or less than about 0.3 mg/day, or less than about 0.2 mg/day. Similarly, selegiline is typically administered at 1.25-2.5 mg per day on maintenance. Sub therapeutic dosages are less than 1.25 mg/day, preferably less than about 1.2 mg/day, or less than about 1.1 mg/day, or less than about 1.0 mg/day, or less than about 0.9 mg/day, or less than about 0.8 mg/day, or less than about 0.7 mg/day, or less than about 0.6 mg/day, or less than about 0.5 mg/day, or less than about 0.4 mg/day.

Dopamine Agonists.

In certain embodiments administration of one or more MAOB inhibitors in conjunction with lithium is contemplated for the treatment or prophylaxis of PD. Dopamine agonists include bromocriptine (e.g., PARLODEL®), cabergoline (e.g., DOSTINEX®), pergolide (e.g., PERMAX®), pramipexole (e.g., MIRAPEX®, SIFROL®), ropinirole (e.g., REQUIP®), piribedil, apomorphine (e.g., APOKYN®), rigotine (e.g., NEUPRO®), quinagolide (e.g., NORPROLAC®), fenoldopam, and lisuride.

In certain embodiments the dopamine agonist(s) can be administered in standard therapeutic dosages for PD. In certain embodiments the dopamine agonist(s) can be administered in a low therapeutic doses.

In certain embodiments it is believed that the low dose lithium can act additively or even synergistically with the dopamine agonist(s). Thus, in certain embodiments it is contemplated that the dopamine agonist(s) can be administered in sub-therapeutic doses.

A typical dosage for bromocriptine (PARLODEL®, CYCLOSET®) is about 1.25 mg-100 mg per day on maintenance. Contemplated subtherapeutic dosages are below 100 mg/day, preferably below about 90 mg/day, or below about 80 mg/day, or below about 70 mg/day, or below about 60 mg/day, or below about 50 mg/day.

Combined Formulations.

In certain other embodiments, use of combined formulations (e.g., formulations comprising lithium and L-DOPA and/or an L-DOPA derivative, and/or a MAOB inhibitor and/or a dopamine agonist, and optionally a COMT inhibitor or a DDCI) are contemplated. In certain embodiments the combined formulation is designed to administer the lithium in low doses (e.g., low therapeutic doses or subtherapeutic doses) and the other active agents as described herein in therapeutic doses, low therapeutic doses, or subtherapeutic doses).

The combined formulations contemplated herein can comprise each of the component agents (e.g., lithium, L-DOPA, etc.) the “native” form or, if desired, in the form of salts, esters, amides, prodrugs, derivatives, and the like, provided the salt, ester, amide, prodrug or derivative is suitable pharmacologically, e.g., effective in the present method(s). Salts, esters, amides, prodrugs and other derivatives of the agents described herein can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Certain addition salts include are halide salts, such as may be prepared using hydrochloric or hydrobromic acids. Conversely, basic salts are prepared in a similar manner using a pharmaceutically acceptable base such as sodium hydroxide, potassium hydroxide, ammonium hydroxide, calcium hydroxide, trimethylamine, or the like. Illustrative basic salts include alkali metal salts, e.g., the sodium salt, and copper salts.

In certain embodiments for the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (e.g., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups which may be present within the molecular structure of the drug (e.g., L-DOPA). The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The combined formulations contemplated herein can be useful for parenteral, topical, oral, nasal (or otherwise inhaled), rectal, or local administration such as by intracerebroventricular pump, by aerosol administration, or transdermally, for prophylactic and/or therapeutic treatment of one or more of the pathologies/indications described herein (e.g., to mitigate the onset, progression, or severity of one or more symptoms of Huntington's disease). In various embodiments the combination formulations are formulated as injectables for injection, delivery via an implanted catheter, and the like. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The components (active agents) comprising the combined formulations described herein of this invention are typically each combined with a separate or the same pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agent(s), or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the combined formulation(s) and on the particular physio-chemical characteristics of the active agent(s) comprising the formulation.

In certain embodiments the excipients are preferably sterile and generally free of undesirable matter. These compositions may be sterilized by conventional, well-known sterilization techniques.

In various embodiments the combined formulations described herein can be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, and/or increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, polymeric micelles, multiple emulsions, self-emulsifying, self-microemulsifying, cyclodextrins and derivatives thereof, and dendrimers.

The combined formulations contemplated herein can comprise each of the component agents (e.g., lithium, L-DOPA, etc.) the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the agents described herein can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Certain addition salts include are halide salts, such as may be prepared using hydrochloric or hydrobromic acids. Conversely, basic salts are prepared in a similar manner using a pharmaceutically acceptable base such as sodium hydroxide, potassium hydroxide, ammonium hydroxide, calcium hydroxide, trimethylamine, or the like. Illustrative basic salts include alkali metal salts, e.g., the sodium salt, and copper salts.

In certain embodiments for the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups which may be present within the molecular structure of the drug (e.g., L-DOPA). The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The combined formulations contemplated herein can be useful for parenteral, topical, oral, nasal (or otherwise inhaled), rectal, or local administration such as by intracerebroventricular pump, by aerosol administration, or transdermally, for prophylactic and/or therapeutic treatment of one or more of the pathologies/indications described herein (e.g., to mitigate the onset, progression, or severity of one or more symptoms of Huntington's disease). In various embodiments the combination formulations are formulated as injectables for injection, delivery via an implanted catheter, and the like. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The components (active agents) comprising the combined formulations described herein of this invention are typically each combined with a separate or the same pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agent(s), or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the combined formulation(s) and on the particular physio-chemical characteristics of the active agent(s) comprising the formulation.

In certain embodiments the excipients are preferably sterile and generally free of undesirable matter. These compositions may be sterilized by conventional, well-known sterilization techniques.

In various embodiments the combined formulations described herein can be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, and/or increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, polymeric micelles, multiple emulsions, self-emulsifying, self-microemulsifying, cyclodextrins and derivatives thereof, and dendrimers.

### Administration.

In various embodiments the methods described herein are applied therapeutically to subjects diagnosed as having Parkinson's disease or prophylactically to subjects identified as at risk for PD.

Various medical organizations have created diagnostic criteria to ease and standardize the diagnostic process, especially in the early stages of the disease. The most widely known criteria come from the UK Parkinson's Disease Society Brain Bank and the US National Institute of Neurological Disorders and Stroke. The PD Society Brain Bank criteria require A) Bradykinesia and at least one of the following: muscular rigidity, 4-6 Hz resting tremor, or postural instability not caused by primary visual, vestibular, cerebellar or proprioceptive dysfunction. B) Features that exclude Parkinson's disease as the cause of Parkinsonism include 1) History of repeated strokes with stepwise progression of parkinsonian features; 2) History of repeated head injury; 3) History of definite encephalitis; 4) Neuroleptic treatment at onset of symptoms; 5) More than 1 affected relative; 5) Sustained remission; 6) Strictly unilateral features after 3 years; 7) Supranuclear gaze palsy; 8) Cerebellar signs; 9) Early severe autonomic involvement; 10) Early severe dementia with disturbances of memory, language and praxis; 11) Babinski's sign; 12) Presence of a cerebral tumour or communicating hydrocephalus on computed tomography scan; 13) Negative response to large doses of levodopa (if malabsorption excluded); or 14) 1-methyl-4-phenyl-1,2,3,6-tetrahydropyridine (MPTP) exposure. C) Features that support a diagnosis of Parkinson's disease (three or more required for diagnosis of definite Parkinson's disease: 1) Unilateral onset; 2) Rest tremor present; 3) Progressive disorder; 4) Persistent asymmetry affecting the side of onset most; 5) Excellent (70-100%) response to levodopa; 6) Severe levodopa-induced chorea; 7) Levodopa response for ?5 years; 8) Clinical course of ?10 years.

Criteria of diagnosis of Parkinson disease (Gelb et al, 1999) commissioned and supported by the Advisory Council of the National Institute of Neurological Disorders and Stroke, US National Institutes of Health are as follows:

Grouping of clinical features of Parkinson's disease according to diagnostic utility are shown in Table 1

TABLE 1
Grouping of features characteristic of Parkinson's disease.

GROUP A: Features characteristic of Parkinson's disease:
Resting tremor;
Bradykinesia;
Rigidity;
Asymmetric onset;
GROUP B: Features suggestive of alternative diagnoses:
Features unusual early in the clinical course;
Prominent postural instability in the first 3 years after symptom
onset;
Freezing phenomena in the first 3 years;
Hallucinations unrelated to medications in the first 3 years;
Dementia preceding motor symptoms or in the first year;
Supranuclear gaze palsy (other than restriction of upward gaze)
or slowing of vertical saccades;
Severe, symptomatic dysautonomia unrelated to medications;
Documentation of a condition known to produce Parkinsonism
and plausibly connected to the patient's symptoms (such as
suitably located focal brain lesions or neuroleptic use within the
past 6 months);

Criteria for POSSIBLE diagnosis of Parkinson's disease include: At least 2 of the 4 features in Group A are present; at least 1 of these is tremor or bradykinesia and either:

None of the features in Group B is present; or

Symptoms have been present for less than 3 years, and none of the features in Group B is present to date; and either:
Substantial and sustained response to levodopa or a dopamine agonist has been documented; or
Patient has not had an adequate trial of levodopa or dopamine agonist.

Criteria for PROBABLE diagnosis of Parkinson's disease include: At least 3 of the 4 features in Group A are present and:
None of the features in Group B is present (note: symptom duration of at least 3 years is needed to meet this requirement); and
Substantial and sustained response to levodopa or a dopamine agonist has been documented.

Criteria for DEFINITE diagnosis of Parkinson's disease include: All criteria for POSSIBLE Parkinson disease are met; and histopathological confirmation of the diagnosis is obtained at autopsy.

In humans, olfactory dysfunction often precedes the onset of cardinal motor (as well as non-motor) symptoms of PD (Fornai et al. (2008) Amyotroph Lateral Scler 9: 123-124). PD-associated olfactory deficits include various degrees of loss in odor detection, discrimination and identification. As such, olfactory deficits may be a potential pre-symptomatic or early diagnostic marker for the disorder and, in certain embodiments the methods described herein are contemplated for use in subjects diagnosed as at risk for PD via olfactory deficits.

In certain embodiments the effectiveness of treatment can be determined by comparing a baseline measure of a parameter of the disease before administration of the agent(s) described herein (e.g., low dose lithium and, optionally one or more additional agents such as L-DOPA) is commenced to the same parameter a one or more time points after administration of the active agent(s). Illustrative parameters that can be measured include one or more of the motor, cognitive, or psychiatric symptoms described above, and/or degree of cell death. Mitigation of one or more of the symptoms and/or a decline or reversal of neural cell death indicates partial, substantial, or full efficacy.

In certain embodiments effectiveness of treatment can be evaluated by comparison to the normative time course of the pathology in typical similarly situated subjects (e.g., similar age, severity at first presentation, and the like).

In all of the methods described herein, appropriate dosages of the active agent(s) (e.g., Li) can readily be determined by those of ordinary skill in the art of medicine by monitoring serum concentrations and/or by monitoring the patient for signs of disease amelioration or inhibition, and increasing or decreasing the dosage and/or frequency of treatment as desired and appropriate.

### EXAMPLES

The following examples are offered to illustrate, but not to limit the claimed invention.

### Example 1

### Neuroprotective Effects of Low Dose Lithium

Lithium administration was initially found to protect against in vitro cell death in a hydrogen peroxide treated, stable human alpha-synuclein expressing dopaminergic cell line recently created by our laboratory. In vivo lithium administration (0.255% lithium chloride) delivered in feed to 9 month old pan-neuronal human alpha-synuclein expressing transgenic mice over a 3 month period was sufficient to prevent accumulation of oxidized/nitrated alpha-synuclein in multiple brain regions previously shown to occur as a consequence of paraquat/maneb administration in these transgenics. This included in the glomerular layer, mitral cells, and the granule cell layer of the olfactory bulb (OB), the striatum (ST), the substantia nigra pars compacta (SNpc), in Purkinje cells of the cerebellum (Cb), and the cortex (see, FIGS. 1-3).

Lithium was found to not only prevent alpha-synuclein accumulation in various brain regions, but also to protect against neuronal cell loss in this model (FIG. 4). Importantly, this dosage has previously been demonstrated to result in sera lithium levels in the low therapeutic range (0.65-0.8 mM) in mice independent of strain with no effect on motor function.

### Example 2

### Investigation of Combination of Lithium and SINEMET® to Reduce L-DOPA-Induced Dyskinesia (LID)

SINEMET® is used in the treatment of Parkinson's disease. SINEMET® comprises a combination of carbidopa and levodopa. Short term side effects for SINEMET® include nausea, vomiting, heart rhythm disturbance, while long term side effects include dyskinesia (involuntary movement), restlessness, and confusion.

Dyskinesia is a movement disorder that consists of effects including diminished voluntary movements and the presence of involuntary movements, similar to tics or chorea. Dyskensia in Parkinson's patients ranges from a slight tremor of the hands to uncontrollable movement of, most commonly, the upper body. In certain presentations, it can also be seen in the lower extremities.

Approximately, 30% of PD patient experience LID after 4-6 yrs of treatment and almost 90% suffer from this complication after 9 yrs

In these experiments the combination of lithium (Li) and SINEMET® was investigated for the ability to reduce L-DOPA induced dyskinesia.

### Methods and Results.

A mouse model of Parkinson's disease (C57BL/6J male mice injected with 1-methyl-4-phenyl-1,2,3,6-tetrahydropyridine (MPTP) (25 mg/ml) for 5 days at 9 months) were treated as follows for 2 months:
1) control food (Saline);
2) Lithium food (0.125% LiCl);
3) SINEMET® IP injection (100 mg/kg L-DOPA+25 mg/kg Carbidopa);

4) Lithium+SINEMET®;

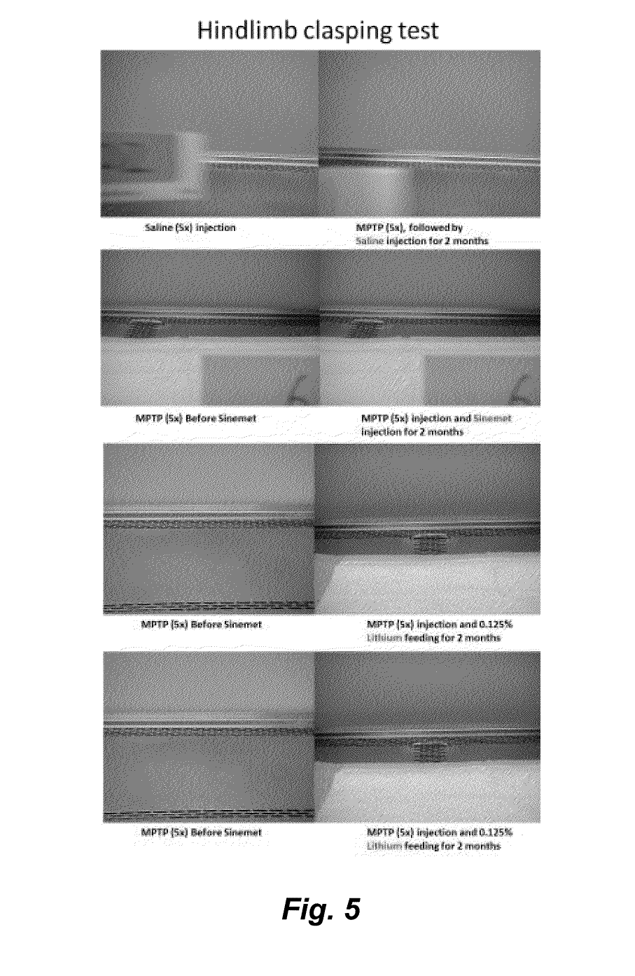

A hind limb clasping test was performed as illustrated in FIG. 5 as summarized below in Table 2.

TABLE 2
Hind limb clasping studies.

| Treatment | Treatment | Shown In |
|---|---|---|
| Saline (5x) injection | MPTP (5x), followed by | FIG. 5, row 1. |
| | Saline injection for 2 months | |
| MPTP (5x) Before | MPTP (5x) injection and | FIG. 5, row 2. |
| SINEMET ® | SINEMET ® injection for 2 | |
| | months | |
| MPTP (5x) Before | MPTP (5x) injection and 0.125% | FIG. 5, row 3. |
| SINEMET ® | Lithium feeding for 2 months | |
| MPTP Before drugs | MPTP (5x) injection, followed by | FIG. 5, row 4. |
| | Lithium-SINEMET ® for 2 | |
| | months | |

The hind limb clasping data provide a measure of abnormal involuntary movement (AIM) in the test animals. Hind limb clasping is a typical AIM in Parkinson's Disease models and is similar to dyskinesia in PD.

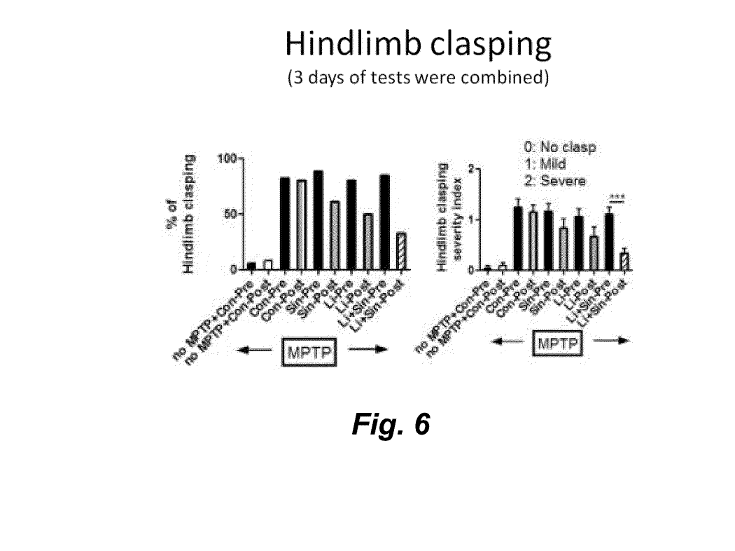

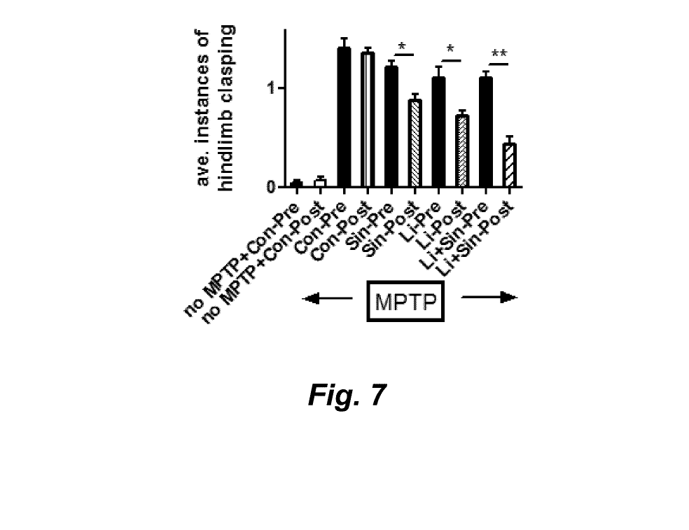

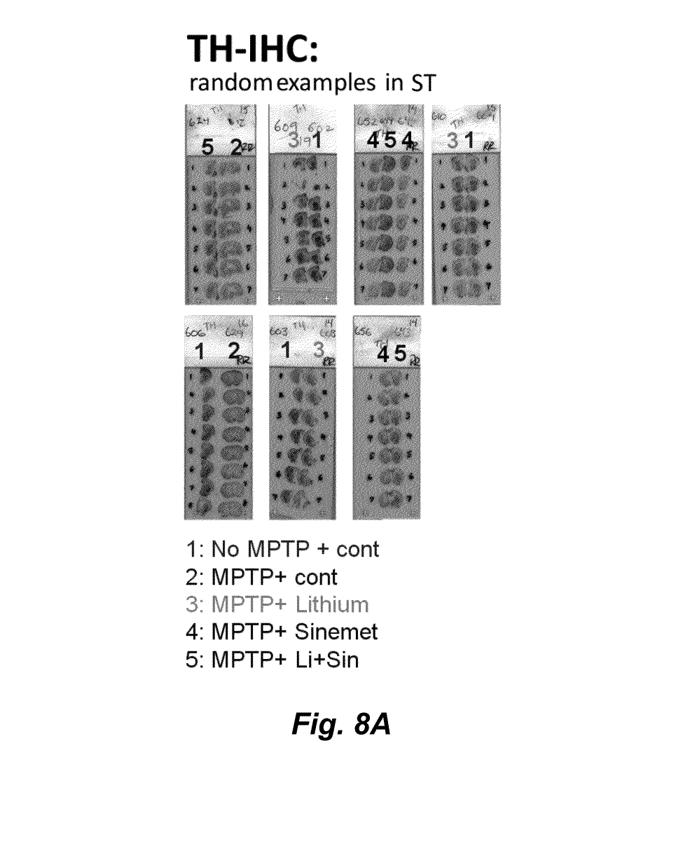

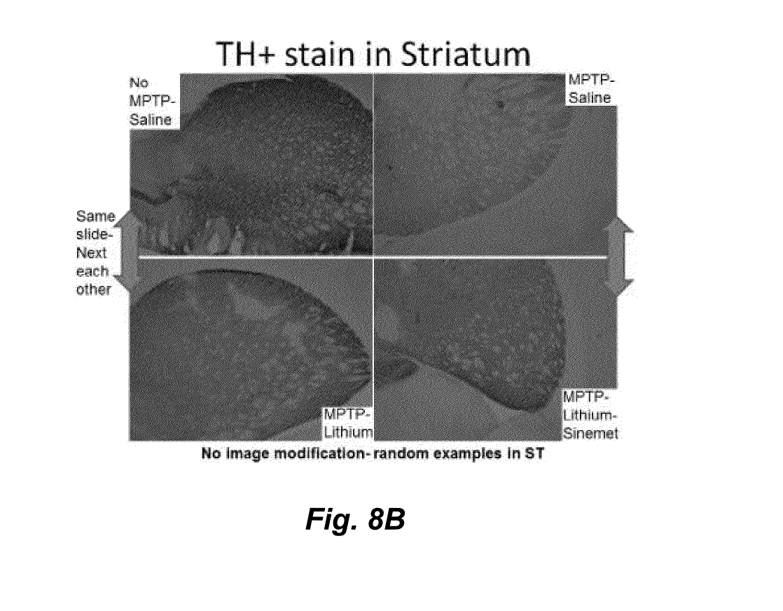

Hindlimb clasping data (% of hindlimb clasping and a hindlimb clasping severity index) are shown in FIG. 6 and an analysis of the hindlimb clasping data is provided in FIG. 7. Use of low dose lithium and a combination of low dose lithium and SINEMET® appears to significantly reduce hindlimb clasping (e.g., Parkinson's associated dyskinesia).

Without being bound by a particular theory it is believed that the protective effects of low dose lithium can be mediated by one or both of two potential mechanisms. In the first mechanism, lithium may suppress 20S proteasomal activity for cytoprotection. In the second mechanism, lithium may up-regulate TH expression in the ST resulting in cell protection against MPTP, mediated by calpain inhibition. These hypotheses were tested.

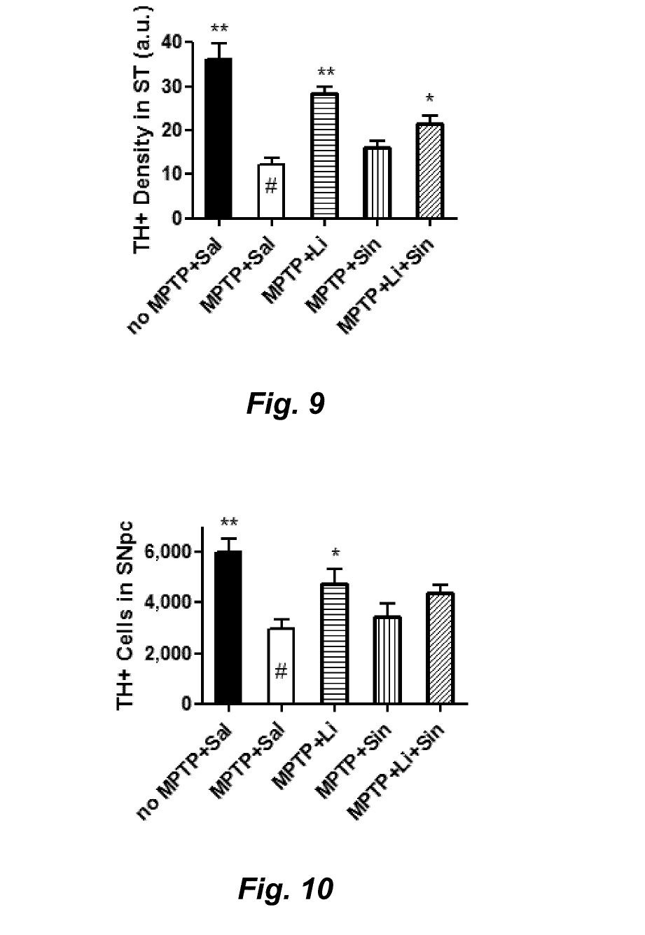

FIG. 9 illustrates the effect of treatment on TH+ density in ST, while FIG. 10 illustrates the effect of treatment on TH+ cell count in substantia nigra pars compacta (SNpc). In both cases, SINEMET® appears to upregulate TH+ expression. Without being bound to a particular theory, it is believed that low dose lithium up-regulates TH levels in the nigrostriatum which acts to attenuate motor deficits as a consequence of decreased dopamine levels in addition to lithium's ability to provide neuroprotection against MPTP neurotoxicity

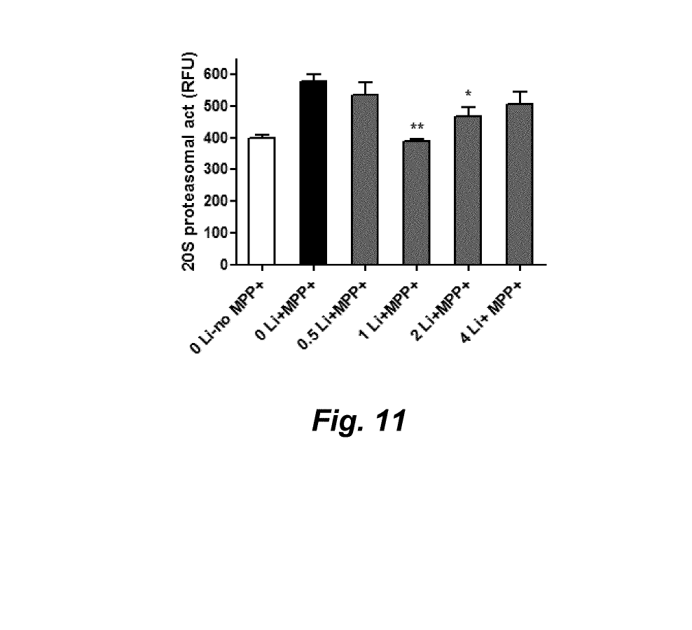

FIG. 11 shows the results of an N27 AT-cell, 20 S proteasomal assay for each of the treatments. As shown in the Figure, lithium appears to suppress 20S proteasomal activity.

Without being bound by a particular theory, it appears that low dose lithium is neuroprotective and when used in conjunction with SINEMET® appears to provide a synergistic improvement in locomotor behavior compared to SINEMET®.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1: A method of delaying the onset of and/or mitigating the severity of one or more symptoms of Parkinson's disease in a mammal, said method comprising:
administering to a mammal diagnosed as having or as at risk for Parkinson's disease a chronic low dose of lithium.

2: The method of claim 1, wherein said low dose is a subtherapeutic dose.

3: The method of claim 1, wherein said low dose is a low therapeutic dosage.

4: The method of claim 1, wherein said low dose is a dosage that results in an average serum concentration below about 0.5 mM.

5-6. (canceled)

7: The method of claim 1, wherein said mammal is a human.

8: The method of claim 7, wherein said human is not under treatment for a neuropsychiatric disorder.

9: The method of claim 7, wherein said human is not under treatment for one or more disorders selected from the group consisting of mania, schizophrenia, bipolar disorder, and psychosis.

10: The method of claim 7, wherein said mammal is a human diagnosed as having Parkinson's disease.

11: The method of claim 7, wherein said mammal is a human diagnosed as at risk for Parkinson's disease

12: The method of claim 7, wherein said human is a human 40 years of age or older.

13-14. (canceled)

15: The method of claim 7, wherein said administration is before the onset of cardinal motor symptoms of Parkinson's disease.

16: The method of claim 7, wherein said administration is to a subject diagnosed as having olfactory dysfunction.

17: The method of claim 7, wherein said human is diagnosed with one or more conditions selected from the group consisting of tremor, bradykinesia, and muscle rigidity.

18: A method of delaying the onset of and/or mitigating the severity of one or more symptoms of Parkinson's disease in a mammal, said method comprising:
administering to a mammal diagnosed as having or as at risk for Parkinson's disease a chronic low dose of lithium in conjunction with a second agent comprising one or more pharmaceutically active agents selected from the group consisting of L-3,4-dihydroxyphenylalanine (L-DOPA) and/or a derivative thereof, a MAOB inhibitor, and a dopamine agonist.

19-22. (canceled)

23: The method of claim 18, wherein said lithium and said second agent are administered in a combined formulation.

24: The method of claim 18, wherein said method further comprises administering said lithium, and L-DOPA in conjunction with a dopa decarboxylase inhibitor and/or a COMT inhibitor and/or pyridoxal phosphate.

25: The method of claim 18, wherein said lithium is administered as a lithium salt, and/or said L-DOP is provided as an L-DOPA ethyl ester.

26. (canceled)

27: The method of claim 18, wherein said L-DOPA is administered in a sub-therapeutic dose for Parkinson's disease.

28: The method of claim 24, wherein said method comprises administering a peripheral DOPA decarboxylase inhibitor (DDCI).

29: The method of claim 28, wherein said DDCI is selected from the group consisting of benserazide, (2S)-3-(3,4-dihydroxyphenyl)-2-hydrazino-2-methylpropanoic acid (carbidopa), and methyldopa.

30: The method of claim 24, wherein said method comprises administering a catechol-O-methyl transferase (COMT) inhibitor.

31: The method of claim 30, wherein said COMT inhibitor is selected from the group consisting of entacapone, tolcapone, and nitecapone.

32: The method of claim 18, wherein said second agent comprises a MAOB inhibitor.

33: The method of claim 32, wherein said MAOB inhibitor comprises one or more agents selected from the group consisting of selegiline, desmethylselegiline, pargyline, rasagiline, AGN-1135, MDL 72195, J 508, lazabemide, milacemide, mofegiline, D-deprenyl, and ladostigil.

34: The method of claim 32, wherein said MAOB inhibitor is administered in a sub-therapeutic dose for Parkinson's disease.

35: The method of claim 18, wherein said second agent comprises a dopamine agonist.

36: The method of claim 35, wherein said dopamine agonist comprises one or more agents selected from the group consisting of bromocriptine, cabergoline, pergolide, pramipexole, ropinirole, piribedil, apomorphine, rigotine, quinagolide, fenoldopam, and lisuride.

37: The method of claim 35, wherein said dopamine agonist is administered in a sub-therapeutic dose for Parkinson's disease.

38. (canceled)

39: The method of claim 18, wherein said low dose is a subtherapeutic dose.

40-43. (canceled)

44: The method of claim 18, wherein said mammal is a human.

45: The method of claim 44, wherein said human is not under treatment for a neuropsychiatric disorder.

46-48. (canceled)

49: The method of claim 44, wherein said human is a human 40 years of age or older.

50-51. (canceled)

52: The method of claim 44, wherein said administration is before the onset of cardinal motor symptoms of Parkinson's disease.

53-54. (canceled)

55: A formulation delaying the onset of and/or mitigating the severity of one or more symptoms of Parkinson's disease said formulation comprising:
lithium;
L-DOPA and/or a derivative thereof; and
one or more pharmaceutically acceptable excipients.

56: The formulation of claim 55, wherein said formulation further comprises a dopa decarboxylase inhibitor and/or a COMT inhibitor, and/or pyridoxal phosphate.

57-58. (canceled)

59: The formulation of claim 56, wherein said formulation comprises a peripheral DOPA decarboxylase inhibitor (DDCI).

60: The formulation of claim 59, wherein said DDCI is selected from the group consisting of benserazide, carbidopa, and methyldopa.

61. (canceled)

62: The formulation of claim 56, wherein said formulation comprises a catechol-O-methyl transferase (COMT) inhibitor.

63: The method of claim 62, wherein said COMT inhibitor is selected from the group consisting of entacapone, tolcapone, and nitecapone.

64. (canceled)
